(19) United States
(12) Patent Application Publication
(10) Pub. No.: US 2026/0272387 A1
(43) Pub. Date: Sep. 17, 2026

(54) RECOGNITION-BASED INFORMATIONAL DIAGNOSTIC DEVICE AND METHODS FOR NON-INVASIVE QUANTUM-INFORMATIONAL MEDICAL DIAGNOSTICS

(71) Applicant: Jonathan Washburn, Austin, TX (US)

(72) Inventor: Jonathan Washburn, Austin, TX (US)

(21) Appl. No.: 19/560,798

(22) Filed: Mar. 9, 2026

Related U.S. Application Data
(60) Provisional application No. 63/770,027, filed on Mar. 11, 2025.

(51) Int. Cl. A61B 5/00

(52) U.S. Cl. CPC A61B 5/7264; A61B 5/0066

(57) ABSTRACT

A non-invasive diagnostic system generates a recognition field and senses a tissue response associated with interaction between the recognition field and biological tissue. A processor computes a nonnegative tissue-state value from one or more coherence-related features derived from the sensed tissue response, computes a bounded recognition metric using a recognition operator R_hat(x)=x/(x+X_opt), and computes a coherence-deviation metric as Delta_R=abs(R_hat_measured−R_hat_healthy) relative to a baseline recognition metric associated with healthy tissue. The system outputs at least one of the coherence-deviation metric and a classification output indicative of tissue state, and in some embodiments generates region-based deviation values to form a deviation map and provides confidence scoring based on signal quality and repeatability.

### CROSS-REFERENCE TO RELATED APPLICATIONS

This application claims the benefit of priority under 35 U.S.C. Section 119(e) to U.S. Provisional Patent Application No. 63/770,027, filed Mar. 11, 2025, the entire disclosure of which is incorporated herein by reference.

### BACKGROUND OF THE INVENTION

Many diagnostic techniques in current clinical practice identify disease only after a sufficient magnitude of structural change, biochemical change, or functional change has developed to produce a detectable signal for a particular modality. In general, the detectability of an abnormality is constrained by modality-specific limits, including spatial resolution, contrast mechanisms, signal-to-noise limitations, assay sensitivity, and the magnitude of physiological or molecular alteration required to cross a diagnostic threshold. As a result, disease processes may begin and progress for a period of time before conventional modalities can reliably detect the associated markers.

Various imaging techniques and clinical procedures can also involve practical and procedural constraints that limit their suitability for frequent screening, early detection, or broad deployment. Certain diagnostic modalities can require large equipment footprints, specialized facilities, trained operators, scheduling delays, and substantial cost. Some procedures can entail patient burden or risk, including exposure to ionizing radiation, use of contrast agents, sedation, or invasive sampling. Invasive techniques such as biopsy can provide clinically valuable tissue characterization, but can carry risks and may not be well-suited to repeated measurements across multiple tissue regions or frequent follow-up screening.

Even within non-invasive modalities, many approaches rely on markers that manifest after disease initiation by a significant interval. For example, structural imaging may rely on growth of a lesion to a size sufficient for detection, and functional imaging may rely on accumulation of tracer uptake patterns or other signatures that can lag underlying initiation and early progression. In neurological, oncological, metabolic, inflammatory, and cardiovascular contexts, detectable structural and biochemical markers may appear only after substantial underlying changes have occurred, potentially reducing the opportunity for earliest-stage detection and intervention.

Non-invasive screening and monitoring can also be constrained by patient-to-patient variability, differences across tissue types, differences across anatomical regions, and measurement variability that can arise from motion, environmental interference, instrument drift, and other non-idealities. These factors can contribute to false positive results, false negative results, indeterminate outcomes, and reduced comparability across devices or clinical settings. Additionally, many conventional diagnostic outputs are not readily expressed as bounded, standardized metrics that are stable across repeated measurements and across patient cohorts, which can complicate longitudinal trending and population-scale screening.

Accordingly, there remains a need for improved diagnostic technologies that can provide earlier detection capability while reducing invasiveness and procedural burden, and that can support repeatable measurements, scalable workflows, and clinically interpretable outputs across tissue types and disease categories.

### SUMMARY OF THE INVENTION

The present disclosure provides non-invasive medical diagnostic devices and methods that measure a tissue response to a generated recognition field and compute a coherence-deviation metric indicative of an abnormal tissue state. In some embodiments, the disclosed devices and methods are configured to detect deviations in informational coherence at an earlier stage than conventional diagnostic modalities that rely on later-arising structural or biochemical markers. In a first aspect, a medical diagnostic system comprises at least one field emitter configured to generate a recognition field according to a defined emission profile, at least one sensor configured to detect at least one response signal from biological tissue in association with the recognition field, at least one processor configured to compute a recognition metric for the tissue from the response signal, at least one baseline representation associated with healthy tissue, and an output interface configured to provide a coherence-deviation metric and/or classification output.

In some embodiments, the processor computes a nonnegative tissue-state scalar x (with x>=0) from one or more sensor signals, and computes a recognition metric R_hat(x) using a bounded, monotone recognition operator of the form:

R_hat(x)=x/(x+X_opt)

In some embodiments, X_opt is a predetermined constant. In one example implementation, X_opt is computed as:

phi=(1+sqrt(5))/2

X_opt=phi/pi

X_opt~=0.5150362148

In some embodiments, the device computes a coherence-deviation metric Delta_R using:

Delta R=abs(R_hat_measured−R_hat_healthy)

In some embodiments, the system classifies a tissue state based on Delta_R relative to one or more thresholds. In one example implementation:

if Delta R>threshold+margin: classification=ABNORMAL

else if Delta R<threshold−margin: classification=HEALTHY

else: classification=INDETERMINATE

In some embodiments, threshold and margin values are selectable per tissue type, anatomical region, patient cohort, device model, clinical setting, or other calibration regime, without changing X_opt.

In some embodiments, the recognition field emitter operates at, or is mapped to, a recognition frequency defined as:

f_rec=c/(X_opt*L_P)

where c is the speed of light and L_P is the Planck length. In some embodiments, an effective recognition frequency, a band-limited approximation, one or more harmonics, sub-harmonics, modulation components, and/or a calibration mapping are used to implement operation in practical hardware while retaining a defined relationship to f_rec.

In some embodiments, the sensor subsystem includes one or more quantum-coherence-sensitive sensors configured to measure an informational response from the tissue. Example sensor types include superconducting quantum interference devices (SQUIDs), quantum photonic sensors, and quantum tunneling junction arrays, as well as other sensors capable of detecting reflected, transmitted, scattered, emitted, induced magnetic, and/or induced electric response signals.

In some embodiments, the system includes a baseline database established from healthy population measurements, where baseline values (e.g., R_hat_healthy and/or distributions thereof) are indexed by tissue type, anatomical region, demographic cohort, and device configuration profile. In some embodiments, the baseline representation is patient-specific such that an initial baseline is measured for an individual and subsequent scans are compared to that individual baseline. In some embodiments, the baseline representation comprises a distribution rather than a single value, and Delta_R is computed relative to that distribution.

In some embodiments, the system computes Delta_R for a plurality of tissue regions and generates a tissue deviation map that spatially localizes coherence deviations for screening, triage, and monitoring. In some embodiments, the system computes a composite score across multiple regions and outputs a risk score and/or recommended follow-up action based on classification results and confidence metrics.

In some embodiments, the system computes a confidence score based on signal quality metrics including signal-to-noise ratio, artifact detection results, sensor health indicators, environmental noise indicators, and/or repeatability across repeated acquisitions. In some embodiments, repeated acquisitions are performed and aggregated to compute a mean deviation metric and a deviation dispersion metric, and classification decisions incorporate confidence intervals derived from measurement dispersion and/or sensitivity analysis.

In some embodiments, the system generates results in real time or near real time. In some embodiments, the system is provided as a handheld, point-of-care device, a portable device, or a stationary clinical system with a scanning structure. In some embodiments, the system integrates with clinical workflows via storage and communication interfaces and can output a structured diagnostic report comprising scan metadata, computed values, classification outputs, and confidence metrics.

In some embodiments, the disclosed systems and methods are applicable across diverse tissue types and disease categories, including oncological, neurological, metabolic, inflammatory, cardiovascular, and other conditions, by utilizing the same coherence-deviation computation framework with tissue- and protocol-specific baseline selection and calibration.

As used herein, “recognition field” refers to a controlled emitted field (e.g., electromagnetic, optical, RF/microwave, terahertz, magnetic, or mixed-mode) defined by an emission profile including one or more waveform parameters. “Coherence-related features” refer to computed signal features indicative of stability and correlation structure of a sensed response relative to a reference and/or relative to itself, including at least one of magnitude-squared coherence, phase stability, correlation decay time, cross-correlation peak magnitude, and band-aggregated coherence. “Baseline representation” refers to a stored value or distribution for healthy tissue for a corresponding acquisition configuration and tissue protocol, including population and/or patient-specific baselines.

### BRIEF DESCRIPTION OF THE DRAWINGS

FIG. 1 illustrates an example diagnostic system comprising a recognition field emitter subsystem, a biological tissue region, a sensor subsystem configured to detect tissue response signals associated with the recognition field, an analysis unit configured to compute recognition metrics and coherence-deviation metrics, a baseline storage subsystem, and an output interface.

FIG. 2 illustrates an example recognition field emitter subsystem comprising a frequency generation module, a waveform shaping module, a power control module, and an emission structure configured to couple a recognition field into a target tissue region.

FIG. 3 illustrates example sensor configurations including (i) a co-located sensor positioned near an emitter structure, (ii) a spatially separated sensor array configured to detect response signals across multiple positions, and (iii) a wrap-around sensor arrangement for partial-body or full-body scanning.

FIG. 4 illustrates an example analysis pipeline comprising signal acquisition, preprocessing, feature extraction, computation of a nonnegative tissue-state scalar, computation of a bounded recognition metric using a recognition operator, computation of a coherence-deviation metric relative to a healthy baseline, classification, confidence scoring, and report generation.

FIG. 5 illustrates example mappings from one or more sensor-derived coherence features to a nonnegative tissue-state scalar x, including transformations configured to ensure x>=0 prior to applying the recognition operator.

FIG. 6 illustrates computation of a coherence-deviation metric Delta_R=abs(R_hat_measured−R_hat_healthy) across multiple tissue regions to generate a deviation map in which each region is associated with a deviation value.

FIG. 7 illustrates an example classification rule set defining HEALTHY, INDETERMINATE, and ABNORMAL zones based on comparison of Delta_R to at least one threshold and a margin band around the threshold.

FIG. 8 illustrates an example scanning workflow comprising patient positioning, selection of a target region, selection of an emission profile, execution of a scan in which the recognition field is emitted and a tissue response is sensed, computation of a coherence-deviation metric and classification output, and optional repeat acquisition based on confidence scoring.

### DETAILED DESCRIPTION

The following description is provided to enable a person of ordinary skill in the art to make and use the disclosed subject matter and to describe multiple embodiments and variations. The disclosed embodiments relate to non-invasive medical diagnostics in which a controlled field is generated, a corresponding tissue response is sensed, and one or more derived metrics are computed, including a bounded recognition metric and a coherence-deviation metric relative to a baseline representation. The embodiments are not limited to a particular tissue type, anatomical region, clinical setting, or sensor modality unless expressly stated.

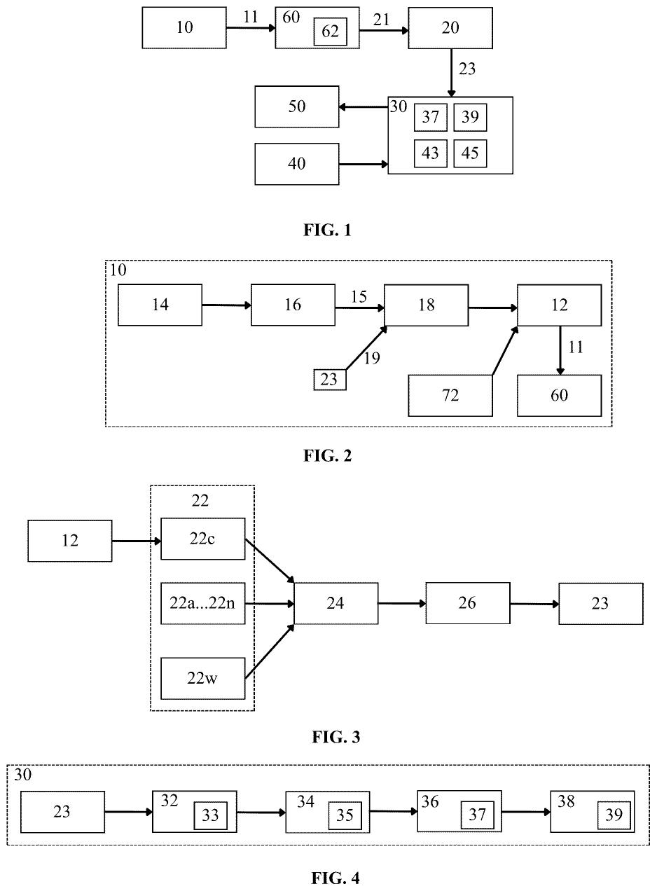

FIG. 1 illustrates an example diagnostic arrangement including a field emitter subsystem 10, a tissue region 60, a sensor subsystem 20, an analysis unit 30, a baseline storage 40, and an output interface 50. In operation, the field emitter subsystem 10 generates a recognition field 11 according to an emission profile and couples the recognition field 11 into or toward the tissue region 60. The tissue region 60 may include a region-of-interest 62 selected by a clinician or operator, by an automated scanning routine, or by a preset anatomical protocol. The sensor subsystem 20 detects one or more tissue response signals 21 associated with interaction between the recognition field 11 and the tissue region 60, and provides one or more sensed signals 23 to the analysis unit 30 for processing. The analysis unit 30 computes at least one tissue-state scalar 37 from the sensed signals 23 and computes a bounded recognition metric 39 and a coherence-deviation metric 43 relative to a baseline value and/or baseline distribution stored in the baseline storage 40. The output interface 50 presents and/or transmits one or more diagnostic outputs including at least the coherence-deviation metric 43 and a classification output 45.

In the illustrated embodiment, the field emitter subsystem 10 includes a frequency generation module 14, a waveform shaping module 16, a power control module 18, and an emission structure 12. The frequency generation module 14 generates one or more carrier and/or modulation components used to form the recognition field 11. The waveform shaping module 16 controls waveform parameters, including at least one of pulse width, repetition rate, duty cycle, chirp, phase coding, amplitude modulation, frequency modulation, and/or polarization control, to produce an emission waveform suitable for inducing measurable response signals in the tissue region 60. The power control module 18 sets and regulates an emission level, and in some embodiments applies closed-loop adjustment based on feedback from the sensor subsystem 20 and/or the analysis unit 30. The emission structure 12 may include one or more antennas, coils, apertures, waveguides, windows, resonators, phased array elements, or other coupling components appropriate to the selected field type and operating band.

In the illustrated embodiment, the sensor subsystem 20 includes one or more sensors 22, an analog front end 24, and an analog-to-digital converter 26. The sensors 22 may be implemented as a single sensor, a pair of sensors, or an array of sensors, and may include one or more coherence-sensitive sensors such as a SQUID-based sensor, a quantum photonic sensor, a quantum tunneling junction array, an interferometric sensor, a magnetometer, an RF receiver, a terahertz receiver, a photodetector, or other sensor capable of detecting at least one response component associated with the tissue response signals 21. The analog front end 24 may include one or more amplifiers, filters, impedance matching networks, mixers, and/or demodulators configured to condition sensor outputs. The analog-to-digital converter 26 digitizes conditioned signals to produce the sensed signals 23 for downstream processing. In some embodiments, the sensor subsystem 20 includes a reference channel 28 corresponding to the emitted waveform and/or a synchronized timing reference, enabling coherent detection, correlation, phase-sensitive detection, and/or computation of coherence functions.

In the illustrated embodiment, the analysis unit 30 includes a preprocessing module 32, a feature extraction module 34, an x-mapping module 36, a recognition metric module 38, a deviation computation module 42, a classification module 44, a confidence module 46, and a report generation module 48. The preprocessing module 32 performs one or more signal conditioning operations, including baseline subtraction, bandpass filtering, notch filtering, denoising, artifact detection, motion compensation, alignment, windowing, normalization, and/or quality gating. The feature extraction module 34 computes one or more coherence-related features 35 from the sensed signals 23, including at least one of spectral coherence, temporal coherence, autocorrelation decay time, cross-correlation peak magnitude, phase stability, amplitude stability, entropy-like measures, and/or other response metrics derived from the tissue response signals 21. The x-mapping module 36 computes the tissue-state scalar 37 as a nonnegative value (x>=0) from one or more coherence-related features 35. The recognition metric module 38 computes the bounded recognition metric 39 by applying a recognition operator to the tissue-state scalar 37.

The deviation computation module 42 computes the coherence-deviation metric 43 as a deviation between the recognition metric 39 and a baseline value and/or baseline distribution retrieved from the baseline storage 40. The classification module 44 computes the classification output 45 based on the coherence-deviation metric 43 and at least one threshold and/or margin rule. The confidence module 46 computes a confidence score 47 based on signal quality, repeatability, dispersion across repeated measurements, and/or other quality indicators. The report generation module 48 generates an output report 49 that may include scan metadata, the coherence-deviation metric 43, the classification output 45, the confidence score 47, and optional supporting values.

In the illustrated embodiment, the baseline storage 40 stores one or more baseline representations including a baseline recognition metric 41 and/or a baseline distribution 41d for at least one tissue category and/or anatomical region. The baseline storage 40 may be local to a device, remote, or hybrid, and may index baseline representations by tissue type, anatomical region, demographic cohort, device configuration profile, emission profile, and/or patient identifier. In some embodiments, the baseline storage 40 stores a patient-specific baseline recognition metric 41 for longitudinal monitoring.

In the illustrated embodiment, the output interface 50 includes a display 52, a communication interface 54, and an output storage 56. The display 52 may present a numerical value of the coherence-deviation metric 43, a classification output 45, and/or a confidence score 47. The communication interface 54 may transmit outputs to an external system, including clinical workflow systems. The output storage 56 may store results locally for audit, trending, or later export.

FIG. 2 illustrates additional detail of an emitter implementation in which the frequency generation module 14 drives the waveform shaping module 16 to produce an emission drive signal 15 that is regulated by the power control module 18 and applied to the emission structure 12 to generate the recognition field 11. In some embodiments, the power control module 18 receives a feedback signal 19 derived from the sensed signals 23 to maintain a target emission intensity, a target signal-to-noise ratio, and/or a target response amplitude. In some embodiments, the field emitter subsystem 10 includes a positioning module 72 configured to control a spatial relationship between the emission structure 12 and the tissue region 60, including distance, angle, and scan position. The positioning module 72 may be manual, mechanical, robotic, or electronically steerable, depending on the emission structure 12.

FIG. 3 illustrates example sensor configurations using the sensors 22. In a first configuration, a co-located sensor 22c is positioned proximate to the emission structure 12 to detect a response signal in a near-field or reflected geometry. In a second configuration, a sensor array 22a through 22n is distributed to detect spatially varying response signals across a region, enabling localization and mapping. In a third configuration, a wrap-around arrangement 22w positions sensors around a portion of a body region to increase capture of transmitted and/or scattered components and to improve robustness against positioning variation. In each configuration, the sensors 22 provide sensor outputs to the analog front end 24 and the analog-to-digital converter 26, yielding the sensed signals 23.

FIG. 4 illustrates an example processing pipeline implemented by the analysis unit 30. The preprocessing module 32 receives the sensed signals 23 and outputs conditioned signals 33. The feature extraction module 34 receives the conditioned signals 33 and outputs coherence-related features 35. The x-mapping module 36 transforms the coherence-related features 35 into the tissue-state scalar 37, with x>=0. The recognition metric module 38 applies a recognition operator to compute the recognition metric 39. In some embodiments, the recognition operator is:

R_hat(x)=x/(x+X_opt)

where X_opt is a predetermined constant. In some embodiments, X_opt is computed as:

phi=(1+sqrt(5))/2

X_opt=phi/pi

X_opt~=0.5150362148

The deviation computation module 42 computes the coherence-deviation metric 43. In some embodiments, the coherence-deviation metric is:

Delta R=abs(R_hat measured−R_hat_healthy)

where R_hat_measured is the recognition metric 39 computed from the tissue-state scalar 37 and R_hat_healthy is the baseline recognition metric 41 retrieved from the baseline storage 40. The classification module 44 computes the classification output 45 using the coherence-deviation metric 43 and at least one threshold 96 and margin 98, and the confidence module 46 computes the confidence score 47. The report generation module 48 generates the output report 49 for presentation on the display 52 and/or transmission via the communication interface 54.

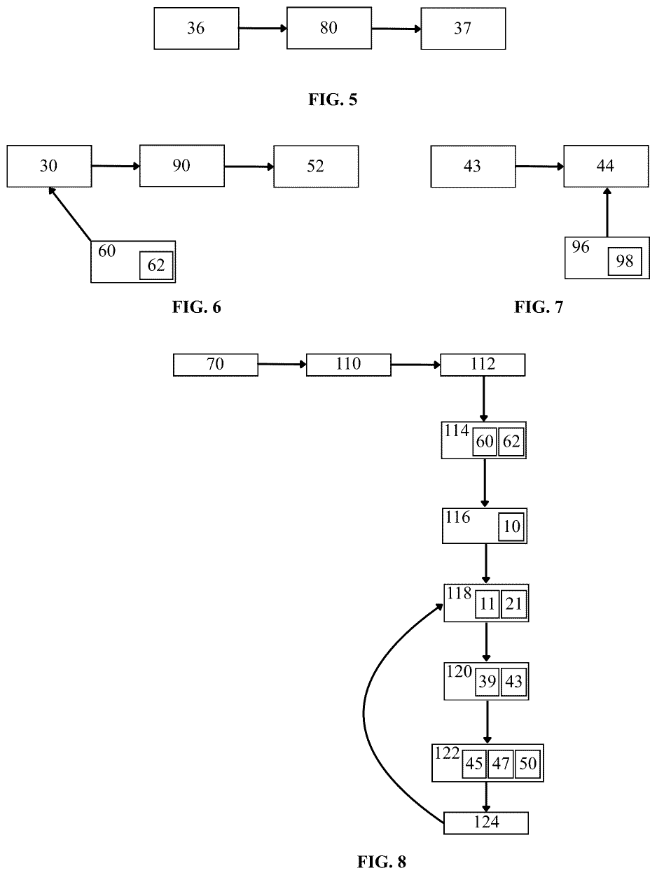

FIG. 5 illustrates example mappings performed by the x-mapping module 36 to ensure that the tissue-state scalar 37 is nonnegative prior to application of the recognition operator. In a first mapping 80, a bounded coherence feature Gamma in (0, 1] is transformed by:

x=(1/Gamma)−1

In a second mapping 82, the bounded coherence feature Gamma in (0, 1] is transformed by:

X=−ln(Gamma)

In a third mapping 84, a baseline feature Gamma_healthy and a measured feature Gamma_measured are transformed by:

X=max(0,(Gamma_healthy−Gamma_measured)/Gamma_healthy)

In a fourth mapping 86, a frequency-domain coherence function gamma (f) is aggregated over a band B using a nonnegative weight w(f) to produce:

x=integral over B of (1−gamma(f)) w(f) df

Each mapping 80, 82, 84, 86 is an example, and other nonnegative transforms and composite mappings may be used.

FIG. 6 illustrates region-based deviation mapping in which the analysis unit 30 computes the coherence-deviation metric 43 for multiple tissue regions 60 and/or multiple regions-of-interest 62 to generate a deviation map 90. In the deviation map 90, each map region 92 is associated with a corresponding deviation value 94 derived from the coherence-deviation metric 43. In some embodiments, the deviation map 90 is presented on the display 52 as a two-dimensional overlay, a heat map, or a segmented region map.

FIG. 7 illustrates an example classification rule set applied by the classification module 44 using the threshold 96 and margin 98. In one embodiment, the classification module 44 determines an ABNORMAL output when the coherence-deviation metric 43 exceeds a threshold 96 plus a margin 98, determines a HEALTHY output when the coherence-deviation metric 43 is below the threshold 96 minus the margin 98, and determines an INDETERMINATE output when the coherence-deviation metric 43 lies within a band defined by the threshold 96 and margin 98. In some embodiments, multiple thresholds 96 are used to define multiple abnormal severity bands. FIG. 8 illustrates an example scanning workflow including a workflow sequence 110 executed by a scan controller 70. In some embodiments, the workflow sequence 110 includes a patient positioning step 112, a region selection step 114 selecting the tissue region 60 and/or the region-of-interest 62, an emission profile selection step 116 configuring the field emitter subsystem 10, a scan execution step 118 in which the recognition field 11 is emitted and the tissue response signals 21 are sensed, an analysis step 120 computing the recognition metric 39 and coherence-deviation metric 43, and an output step 122 providing the classification output 45 and the confidence score 47 via the output interface 50. In some embodiments, the scan controller 70 includes a repeat logic step 124 that triggers one or more repeated acquisitions when the confidence score 47 is below a threshold and/or when an INDETERMINATE classification output is produced.

In the foregoing description, specific reference numerals have been provided for elements shown in FIGS. 1-8 to enable straightforward figure construction and consistent usage throughout the remainder of the specification. The embodiments described herein may be modified, combined, and varied without departing from the scope of the disclosure, and the particular arrangements shown in the figures are provided as non-limiting examples.

In some embodiments, the recognition field 11 generated by the field emitter subsystem 10 is defined by an emission profile 13 stored in the scan controller 70 and/or in the analysis unit 30. The emission profile 13 may specify one or more waveform parameters including a carrier frequency 13a, a modulation frequency 13b, a bandwidth 13c, a pulse width 13d, a repetition rate 13e, a duty cycle 13f, an amplitude 13g, a phase code 13h, a chirp slope 13i, a polarization state 13j, and/or a spatial distribution parameter 13k that controls a beam shape, beam steering, or emission direction. In some embodiments, the emission profile 13 further specifies a scan trajectory 13m defining a sequence of positions and/or orientations for the emission structure 12 and/or the sensors 22, enabling region-based mapping as described with respect to the deviation map 90.

In some embodiments, the frequency generation module 14 includes a reference oscillator 14a, a synthesizer 14b, and a modulation generator 14c. The reference oscillator 14a may be a temperature-compensated crystal oscillator, an oven-controlled oscillator, an atomic reference, or another stable timing source. The synthesizer 14b may generate one or more programmable frequencies used as the carrier frequency 13a and/or the modulation frequency 13b, and may include a phase-locked loop, direct digital synthesis, or another frequency generation technique. The modulation generator 14c may generate one or more modulation waveforms including sinusoidal, square, pseudo-random, spread-spectrum, chirped, or coded sequences. In some embodiments, the waveform shaping module 16 includes a pulse former 16a, a phase coder 16b, a windowing function module 16c, and a harmonic shaping module 16d that collectively control spectral content and time-domain structure of the emission drive signal 15.

In some embodiments, the power control module 18 includes an amplifier stage 18a, a power monitor 18b, and a limiter 18c. The amplifier stage 18a drives the emission structure 12 to produce the recognition field 11. The power monitor 18b measures an emitted power estimate 18d and provides the emitted power estimate 18d to the scan controller 70 and/or the analysis unit 30. The limiter 18c enforces a maximum emission limit 18e. In some embodiments, the maximum emission limit 18e is set based on an operating mode 18f, a tissue type 18g, a target depth 18h, and/or a patient safety profile 18i. In some embodiments, the scan controller 70 automatically reduces emission amplitude 13g, reduces duty cycle 13f, or pauses emission when the emitted power estimate 18d exceeds the maximum emission limit 18e or when a temperature sensor 18j indicates an over-temperature condition.

In some embodiments, the emission structure 12 is selected based on the intended operating band and coupling geometry. For example, when the recognition field 11 includes an RF or microwave component, the emission structure 12 may include a coil, loop, patch antenna, dipole, horn, waveguide, or phased array elements. When the recognition field 11 includes an optical component, the emission structure 12 may include an optical window, lens, fiber output, photonic emitter, or collimation assembly. When the recognition field 11 includes a terahertz component, the emission structure 12 may include a terahertz antenna, photoconductive switch emitter, or resonant structure. In some embodiments, the emission structure 12 includes a coupling interface 12a configured to maintain a stable standoff distance 12b between the emission structure 12 and a skin surface 61 adjacent the tissue region 60, thereby improving repeatability of the measured tissue response signals 21.

In some embodiments, the recognition field 11 is defined to be “recognition-referenced” in the sense that the emission profile 13 is selected using at least one recognition parameter derived from a predetermined constant X_opt. In some embodiments, a recognition frequency parameter is defined as:

f_rec=c/(X_opt*L_P)

where c is the speed of light and L_P is the Planck length. In some embodiments, the system does not directly generate a waveform at f_rec, and instead defines an effective recognition mapping 17 that maps f_rec to a practical operating band. In some embodiments, the effective recognition mapping 17 produces one or more effective operating frequencies 17a, bands 17b, and/or modulation components 17c such that the emitted waveform maintains a defined relationship to f_rec while being implementable in practical hardware. For example, in some embodiments, the effective recognition mapping 17 includes one or more scaling operations, mixing operations, frequency division operations, frequency multiplication operations, harmonic selection operations, and/or band-limited approximations to generate one or more carrier and modulation components for the emission profile 13.

By way of non-limiting example, an effective recognition mapping may map a recognition-referenced parameter to a permitted device band by selecting a carrier frequency in a permitted band, applying a modulation component derived from a scaled or divided form of a recognition-referenced parameter, and using coherent detection relative to a reference channel to compute coherence-related features. In such embodiments, the defined relationship may be implemented as a stored mapping table or scaling rule applied deterministically by the scan controller.

In some embodiments, the tissue response signals 21 include one or more components such as reflected components 21a, transmitted components 21b, scattered components 21c, induced magnetic components 21d, induced electric components 21e, and/or emitted components 21f associated with tissue interaction with the recognition field 11. In some embodiments, the sensor subsystem 20 is configured for coherent detection by using the reference channel 28 as a phase and/or timing reference corresponding to the emission drive signal 15. In some embodiments, the reference channel 28 includes a sampled version of the emission drive signal 15 and/or a timing marker 28a synchronized to pulse edges, coded sequence boundaries, or modulation cycles.

In some embodiments, the sensors 22 include a quantum-coherence-sensitive sensor 22q, such as a SQUID-based sensor, a quantum photonic sensor, or a quantum tunneling junction array. In some embodiments, the quantum-coherence-sensitive sensor 22q is used to detect small variations in one or more response components attributable to coherence changes in the tissue response signals 21. In other embodiments, the sensors 22 include one or more conventional sensors 22r, such as magnetometers, RF receivers, photodetectors, or interferometric receivers, and the analysis unit 30 computes coherence-related features 35 from the sensed signals 23 using signal processing methods described herein. In some embodiments, one or more of the sensors 22 are arranged as the sensor array 22a through 22n and the analysis unit 30 uses differences across sensor channels to infer spatial localization for the deviation map 90.

In some embodiments, the analog front end 24 includes a low-noise amplifier 24a, a bandpass filter 24b matched to an operating band of the emission profile 13, and a demodulator 24c configured to produce an in-phase component 24d and a quadrature component 24e. In such embodiments, the analog-to-digital converter 26 samples the in-phase component 24d and quadrature component 24e to produce a complex-valued sensed signal 23c used by the feature extraction module 34 to compute phase stability, amplitude stability, and coherence measures. In other embodiments, the analog front end 24 includes a lock-in amplifier 24f synchronized to a modulation component 13b and the analog-to-digital converter 26 digitizes a lock-in output 24g representing a coherent response magnitude.

In some embodiments, the preprocessing module 32 identifies and mitigates artifacts in the sensed signals 23. For example, in some embodiments the preprocessing module 32 includes an artifact detector 32a configured to identify motion artifacts 32b, power-line interference artifacts 32c, saturation artifacts 32d, coupling variation artifacts 32e, and/or environmental interference artifacts 32f. In some embodiments, the artifact detector 32a produces an artifact flag 32g and an artifact severity score 32h. In some embodiments, the preprocessing module 32 applies an artifact mitigation operation 32i, such as notch filtering, adaptive filtering, interpolation, window selection, segment rejection, or re-alignment, to produce the conditioned signals 33. In some embodiments, the scan controller 70 triggers the repeat logic step 124 when the artifact severity score 32h exceeds a repeat threshold 32j.

In some embodiments, the feature extraction module 34 computes one or more coherence-related features 35 that are designed to be comparable across acquisitions and robust to amplitude scale differences. In some embodiments, the feature extraction module 34 computes a magnitude-squared coherence feature gamma (f) in a frequency domain between a reference signal derived from the reference channel 28 and a measured response derived from the conditioned signals 33. In some embodiments, the feature extraction module 34 computes a band-aggregated coherence feature Gamma by integrating or averaging gamma (f) over a frequency band B defined by the emission profile 13 and/or the effective operating band 17b. In some embodiments, the feature extraction module 34 computes a temporal coherence feature 35t by measuring a correlation decay time, phase drift, or stability of the complex response over time. In some embodiments, the feature extraction module 34 computes a phase stability feature 35p as a variance or dispersion of phase across repeated measurements.

In some embodiments, the x-mapping module 36 computes the tissue-state scalar 37 using one or more nonnegative transforms selected from the mappings 80, 82, 84, 86 described with respect to FIG. 5. In some embodiments, the x-mapping module 36 selects a mapping based on a mapping selection rule 88 stored in the scan controller 70 and/or the baseline storage 40. The mapping selection rule 88 may depend on a tissue type identifier 60t, an anatomical region identifier 60r, an emission profile identifier 13id, and/or a sensor configuration identifier 22id. In some embodiments, the x-mapping module 36 produces not only a scalar value x but also an x-quality indicator 37q representing uncertainty, stability, or confidence in the computed tissue-state scalar 37.

In some embodiments, the recognition metric module 38 computes the recognition metric 39 using the recognition operator:

R_hat(x)=x/(x+X_opt)

and stores the computed value as R_hat_measured 39m. In some embodiments, the recognition metric module 38 also computes a sensitivity value 39s indicating how changes in x affect R_hat. In some embodiments, the sensitivity value 39s is computed using:

dR_hat/dx=X_opt/(x+X_opt)\hat{ }2

and is used by the confidence module 46 to propagate uncertainty from the x-quality indicator 37q into an uncertainty for R_hat_measured 39m.

In some embodiments, the baseline storage 40 stores baseline recognition metrics 41 as values and/or distributions. In a value-based baseline implementation, the baseline recognition metric 41 includes R_hat_healthy 41h for a corresponding tissue type and region, and the deviation computation module 42 computes:

Delta R=abs(R_hat_measured−R_hat_healthy)

In a distribution-based baseline implementation, the baseline distribution 41d includes at least one of a mean 41m, a standard deviation 41s, a quantile set 41q, or another statistical representation, and the deviation computation module 42 computes a distribution-relative deviation 43d such as a z-score deviation 43z or a quantile deviation 43qv that is optionally combined with Delta_R. In some embodiments, a patient-specific baseline 41p is stored for a patient identifier 60p and the deviation computation module 42 computes deviation relative to the patient-specific baseline 41p for longitudinal monitoring.

In some embodiments, the classification module 44 computes the classification output 45 using the threshold 96 and margin 98 as described with respect to FIG. 7. In some embodiments, the threshold 96 and margin 98 are retrieved from a classification policy 97 stored in the baseline storage 40 and indexed by tissue type identifier 60t and region identifier 60r. In some embodiments, the classification policy 97 defines multiple thresholds 96 to define severity bands, including a first abnormal threshold 96a and a second abnormal threshold 96b, and the classification output 45 includes a severity label 45s. In some embodiments, the classification module 44 computes an indeterminate condition when the coherence-deviation metric 43 lies within a margin band, and the scan controller 70 triggers the repeat logic step 124 to obtain additional acquisitions in the same region-of-interest 62 and/or using a second emission profile. In some embodiments, the confidence module 46 computes the confidence score 47 based on at least one of: a signal-to-noise estimate 46a, the artifact severity score 32h, a repeatability estimate 46b across repeated acquisitions, a dispersion estimate 46c (e.g., standard deviation of repeated Delta_R values), and a sensor health status 46d. In some embodiments, the scan controller 70 performs N repeated acquisitions, producing Delta_R_i values for i=1 . . . . N, and the confidence module 46 computes a mean deviation 46m and a deviation dispersion 46s. In some embodiments, the confidence module 46 produces a confidence interval 46i for Delta_R and/or for R_hat_measured, and the classification module 44 uses the confidence interval 46i to reduce false classifications, including requiring that a lower bound of the confidence interval exceed a threshold for an ABNORMAL classification.

In some embodiments, the deviation map 90 described with respect to FIG. 6 is generated by associating deviation values 94 with scan positions 93. In some embodiments, the scan positions 93 correspond to positions and/or orientations set by the positioning module 72, and the scan controller 70 records a position identifier 93id for each acquisition. In some embodiments, the deviation map 90 includes a region segmentation 90s that groups map regions 92 into anatomical zones, and the report generation module 48 outputs both per-region deviation values and zone-aggregated deviation values.

In some embodiments, the workflow sequence 110 described with respect to FIG. 8 includes storing scan metadata 49m in the output report 49. The scan metadata 49m may include an emission profile identifier 13id, a sensor configuration identifier 22id, a timestamp 49t, a region identifier 60r, and a baseline identifier 41id identifying which baseline representation was used. In some embodiments, the report generation module 48 stores the computed tissue-state scalar 37, the recognition metric 39, the coherence-deviation metric 43, the classification output 45, and the confidence score 47, thereby enabling audit, trending, and re-analysis.

In some embodiments, the output interface 50 provides an operator guidance output 58. For example, the operator guidance output 58 may prompt repositioning when a coupling variation artifact 32e is detected, may prompt reduced motion when motion artifacts 32b are detected, and/or may prompt a repeat scan when an INDETERMINATE classification output is produced. In some embodiments, the operator guidance output 58 includes a stability indicator 58s indicating whether the measured response has reached a stability condition suitable for computation of x and Delta_R.

In some embodiments, the diagnostic system described herein is implemented as a portable device, a handheld device, or a stationary clinical device, without altering the computational framework described with respect to the analysis unit 30. In some embodiments, portions of the analysis unit 30 are implemented in firmware and portions are implemented in software. In some embodiments, a portion of computation is performed remotely, where the communication interface 54 transmits at least one of the sensed signals 23, the conditioned signals 33, the coherence-related features 35, or intermediate metrics (including x or R_hat) to an external computing system, and receives a report output that is stored as the output report 49.

The foregoing paragraphs describe non-limiting embodiments and provide reference numerals intended to support figure construction for FIGS. 1-8. Additional embodiments may include variations in emission profiles, sensor types, mapping rules, baseline policies, classification policies, and workflow policies, while retaining the core computation of a nonnegative tissue-state scalar 37, a bounded recognition metric 39 computed using R_hat(x)=x/(x+X_opt), and a coherence-deviation metric 43 computed relative to a baseline representation.

In some embodiments, the scan controller 70 implements a baseline selection policy 74 that selects which baseline representation in the baseline storage 40 is used for a given scan. In one embodiment, the baseline selection policy 74 selects a population baseline 41c based on a tissue type identifier 60t and a region identifier 60r. In another embodiment, the baseline selection policy 74 selects a patient-specific baseline 41p based on a patient identifier 60p and the region identifier 60r, enabling longitudinal trending for the same individual. In some embodiments, when a patient-specific baseline 41p is not available, the baseline selection policy 74 defaults to the population baseline 41c and stores the first measured baseline recognition metric 41n as a patient-specific baseline candidate for later use.

In some embodiments, the baseline storage 40 stores multiple baseline entries for different emission profile identifiers 13id and/or different sensor configuration identifiers 22id, such that baseline comparisons are made within the same acquisition configuration. In such embodiments, the baseline selection policy 74 selects a baseline entry having a matching emission profile identifier 13id and sensor configuration identifier 22id. In some embodiments, when an exact match is not available, the baseline selection policy 74 selects a nearest baseline entry using a similarity rule 74s that compares one or more of the carrier frequency 13a, bandwidth 13c, modulation frequency 13b, and sensor type of the sensors 22. In some embodiments, the similarity rule 74s enforces a compatibility constraint 74c that prevents using baselines derived from materially different acquisition modes.

In some embodiments, the scan controller 70 implements a calibration workflow 75 that is executed prior to a patient scan and/or periodically during device operation. In one embodiment, the calibration workflow 75 includes acquiring a calibration measurement from a calibration target 76 positioned in a known relationship with the emission structure 12 and the sensors 22. The calibration target 76 may be a phantom, a reference material, or an engineered structure configured to produce a stable response signature. In some embodiments, the analysis unit 30 computes a calibration response metric 77 from the sensed signals 23 and stores a calibration profile 78 in the baseline storage 40. In some embodiments, the calibration profile 78 includes a gain correction 78g, a phase correction 78p, a noise-floor estimate 78n, and/or a bandpass correction 78b, and the preprocessing module 32 applies the calibration profile 78 to produce the conditioned signals 33.

In some embodiments, the scan controller 70 implements a region scan plan 71 that defines a sequence of scan positions 93 for constructing the deviation map 90. In one embodiment, the region scan plan 71 defines a grid of scan positions 93 over a target anatomical region, and each scan position 93 corresponds to a distinct region-of-interest 62. In some embodiments, the scan controller 70 records a position coordinate 93x and 93y for each scan position 93 and associates the position coordinate 93x and 93y with the deviation value 94 computed for the corresponding region-of-interest 62. In some embodiments, the scan controller 70 further records a distance value 93d representing the standoff distance 12b between the emission structure 12 and the skin surface 61, and the confidence module 46 reduces the confidence score 47 when the distance value 93d deviates beyond a distance tolerance 93t.

In some embodiments, the scan controller 70 implements an adaptive scan policy 73 that modifies the region scan plan 71 based on intermediate results. For example, in some embodiments, when an initial deviation value 94 at a scan position 93 exceeds a refinement threshold 73a, the adaptive scan policy 73 inserts additional scan positions 93 in a neighborhood around the scan position 93 to increase spatial resolution of the deviation map 90 in a suspected abnormal region. In some embodiments, the adaptive scan policy 73 increases a dwell time 73d or increases the number of repeated acquisitions N at scan positions associated with higher deviation values 94, thereby increasing confidence for localized abnormalities.

In some embodiments, the preprocessing module 32 and/or the confidence module 46 incorporate environmental compensation using one or more environmental sensors 79. The environmental sensors 79 may include an ambient electromagnetic sensor 79e, a temperature sensor 79t, a humidity sensor 79h, and/or an accelerometer 79a. In some embodiments, the preprocessing module 32 uses an ambient noise estimate 79n to apply adaptive filtering and reduce environmental interference in the conditioned signals 33. In some embodiments, the confidence module 46 reduces the confidence score 47 when the ambient noise estimate 79n exceeds a noise threshold 79x or when the accelerometer 79a indicates motion above a motion threshold 79m.

In some embodiments, the feature extraction module 34 computes coherence-related features 35 using multiple methods and selects one or more features using a feature selection rule 34s. For example, in some embodiments, the feature extraction module 34 computes a spectral coherence feature 35f over a band B, a temporal coherence feature 35t based on correlation decay, and a phase stability feature 35p based on phase variance. In some embodiments, the x-mapping module 36 computes the tissue-state scalar 37 using a weighted combination of multiple deviation features, such as:

x=a1*d1+a2*d2+a3*d3

where a1, a2, and a3 are nonnegative weights and d1, d2, and d3 are nonnegative deviation measures derived from the coherence-related features 35. In some embodiments, the weights are stored as mapping weights 36w in the baseline storage 40 and are selected based on the tissue type identifier 60t and the region identifier 60r.

In some embodiments, the analysis unit 30 supports a multi-profile scan mode 81 in which two or more emission profiles 13 are used during a scan session for the same tissue region 60. In one embodiment, the multi-profile scan mode 81 includes a first emission profile 13(1) and a second emission profile 13(2) having different carrier frequencies 13a or different modulation frequencies 13b, and the analysis unit 30 computes a first deviation value 94(1) and a second deviation value 94(2). In some embodiments, the classification module 44 computes the classification output 45 using a fusion rule 83 that combines deviation values across profiles. For example, in one embodiment, the fusion rule 83 determines ABNORMAL when either deviation value exceeds a threshold, and in another embodiment, the fusion rule 83 determines ABNORMAL when a weighted combination exceeds a threshold. In some embodiments, multi-profile operation improves robustness to tissue heterogeneity and improves discrimination for certain tissue types.

In some embodiments, the classification module 44 implements a multi-stage decision logic 85 that includes a screening stage 85a and a refinement stage 85b. In the screening stage 85a, the classification module 44 uses a screening threshold 96s and a screening margin 98s to classify a region as HEALTHY, INDETERMINATE, or ABNORMAL. In the refinement stage 85b, which may be triggered for INDETERMINATE or ABNORMAL results, the scan controller 70 executes additional acquisitions, optionally with an alternative emission profile, and the classification module 44 uses a refinement threshold 96r and refinement margin 98r to produce a final classification. In some embodiments, the refinement stage 85b reduces false positives and improves confidence for early screening contexts.

In some embodiments, the analysis unit 30 computes a longitudinal trend output 87 based on repeated scans over time. In one embodiment, the baseline storage 40 stores a sequence of prior deviation values 94 over time for a patient identifier 60p, and the analysis unit 30 computes a trend metric 87t representing a change in deviation values across time points. In some embodiments, the output report 49 includes the trend metric 87t and an alert flag 87a indicating whether a trend exceeds a trend threshold 87x. In some embodiments, trend monitoring supports therapy response monitoring and early warning for progression.

In some embodiments, the system includes a safety controller 89 coupled to the power control module 18. The safety controller 89 may enforce one or more exposure limits 89e by limiting emitted power, duty cycle, and/or scan duration. In some embodiments, the safety controller 89 enforces a maximum scan duration 89d for a given region, a maximum repetition rate 13e, and/or a maximum amplitude 13g. In some embodiments, the safety controller 89 disables emission when a fault condition 89f is detected, including amplifier faults, sensor faults, over-temperature conditions, or enclosure faults. In some embodiments, the output interface 50 presents a safety status indicator 89s indicating whether the device is operating within specified limits.

In some embodiments, the report generation module 48 generates the output report 49 in a structured format 49s suitable for storage and transmission. In some embodiments, the structured format 49s includes a scan record 49r storing scan metadata 49m and results fields including the tissue-state scalar 37, the recognition metric 39, the baseline recognition metric 41, the coherence-deviation metric 43, the classification output 45, and the confidence score 47. In some embodiments, the structured format 49s further includes a region map record 49q storing deviation values 94 for multiple scan positions 93 and a map visualization field 49v used by the display 52 to render the deviation map 90.

In some embodiments, the communication interface 54 transmits outputs to an external computing system 57. In one embodiment, the external computing system 57 stores results in a patient record database 57p and provides a clinician review interface 57c. In another embodiment, the external computing system 57 performs at least a portion of the analysis steps, including computation of x, computation of R_hat_measured, computation of Delta_R, and/or classification, and transmits a computed report back to the device for presentation. In some embodiments, the device transmits only coherence-related features 35 to reduce bandwidth and to reduce transmission of raw sensor signals.

In some embodiments, the analysis unit 30 is implemented by a processor 30p and a memory 30m storing instructions that cause the analysis unit 30 to perform the operations described herein. The processor 30p may be a microcontroller, CPU, DSP, FPGA, GPU, or combinations thereof. In some embodiments, the preprocessing module 32 and feature extraction module 34 are implemented in hardware logic or firmware to support real-time processing, and the classification module 44 and report generation module 48 are implemented in software.

In some embodiments, one or more steps described herein are implemented as instructions stored on a non-transitory computer-readable medium 30c. In such embodiments, execution of the instructions by the processor 30p causes the system to perform operations including controlling the field emitter subsystem 10, acquiring sensed signals 23, computing coherence-related features 35, computing the tissue-state scalar 37, computing the recognition metric 39 using R_hat(x)=x/(x+X_opt), computing the coherence-deviation metric 43 relative to a baseline representation, and outputting a classification output 45.

In some embodiments, the analysis unit 30 is implemented by a computing device that includes one or more interfaces configured to receive digitized sensor data from the sensor subsystem 20 and to provide outputs to external systems. Example interfaces include a wired interface, wireless interface, removable media interface, display interface, and/or network interface for transmitting a structured report including scan metadata, computed metrics, classification output, and confidence information.

In some embodiments, the disclosed diagnostic framework is applied to diverse tissue types and anatomical regions without changing the core computation pathway. For example, the system may be configured for a first tissue protocol 91a and a second tissue protocol 91b, where each tissue protocol selects a corresponding emission profile 13, mapping selection rule 88, baseline selection policy 74, and classification policy 97. In some embodiments, the system supports screening, triage, monitoring, and longitudinal assessment by using consistent computation of Delta_R relative to a baseline and by outputting both region-level deviation values and aggregated outputs.

The foregoing embodiments are provided to illustrate example implementations and are not intended to limit the scope of the disclosure. A person of ordinary skill in the art will recognize that variations may be made in field generation, sensing modality, preprocessing, feature extraction, mapping to the tissue-state scalar 37, baseline policies, classification logic, and workflow policies, while retaining the core functionality of computing a bounded recognition metric 39 and a coherence-deviation metric 43 relative to a baseline representation and outputting a non-invasive diagnostic result.

In some embodiments, the system further supports multi-region aggregation to produce an overall screening output for a patient scan session. In one implementation, the analysis unit 30 computes deviation values 94 for a plurality of scan positions 93 and computes an aggregated deviation score 101. The aggregated deviation score 101 may be computed as a maximum deviation 101a, a weighted sum deviation 101b, a percentile deviation 101c, or another aggregation across a set of map regions 92. In one embodiment, the aggregated deviation score 101 is computed as:

aggregated_score=max over regions of (Delta_R_region)

In another embodiment, the aggregated deviation score 101 is computed as:

aggregated_score=sum over regions i of (w_i*Delta_R_i)

where w_i are nonnegative region weights that may be determined based on clinical importance of specific subregions, expected disease prevalence patterns, or scan protocol requirements. In some embodiments, the output interface 50 presents the aggregated deviation score 101 together with region-level deviation values 94 to support clinician interpretation.

In some embodiments, the analysis unit 30 computes a region confidence score 102 for each map region 92. In one embodiment, the region confidence score 102 incorporates the artifact severity score 32h, a region SNR estimate 46a, a region repeatability estimate 46b, and a standoff stability estimate 102d based on the distance value 93d. In some embodiments, the output interface 50 displays a confidence overlay 1020 on the deviation map 90, and the classification module 44 suppresses or flags classifications for regions having a confidence below a confidence threshold 102t.

In some embodiments, the scan controller 70 implements a repeat acquisition rule 103 that determines when to repeat scans and how many repeats to perform. In one embodiment, the repeat acquisition rule 103 triggers repetition when the classification output 45 is INDETERMINATE. In another embodiment, the repeat acquisition rule 103 triggers repetition when the artifact severity score 32h exceeds the repeat threshold 32j, when the confidence score 47 is below a confidence threshold 103c, and/or when the standoff distance 12b changes beyond the distance tolerance 93t. In some embodiments, the repeat acquisition rule 103 defines a maximum repeat count 103m and a maximum time budget 103b for repeated scans to control workflow time.

In some embodiments, the analysis unit 30 supports a baseline update policy 104. In one embodiment, the baseline update policy 104 updates a patient-specific baseline 41p when a clinician confirms a HEALTHY outcome and when the confidence score 47 exceeds an update threshold 104t. In such embodiments, the baseline update policy 104 may store an updated baseline value 41u as a weighted blend:

R_hat_patient_new=(1−alpha)*R_hat_patient_old+alpha*R_hat_measured

where alpha is an update weight in (0, 1] selected based on time since prior baseline and measurement confidence. In some embodiments, the baseline update policy 104 stores multiple prior values and computes an updated baseline using a robust estimator (e.g., median of recent healthy-confirmed measurements) to reduce the effect of outliers.

In some embodiments, the system provides an operator selectable scan mode 105. In one embodiment, the operator selectable scan mode 105 includes a screening mode 105a, a diagnostic mode 105b, and a monitoring mode 105c. In screening mode 105a, the scan controller 70 selects a shorter scan duration and a subset of scan positions 93 to provide a rapid output. In diagnostic mode 105b, the scan controller 70 selects more scan positions and/or longer dwell times, and may apply multi-profile scanning to increase discrimination. In monitoring mode 105c, the scan controller 70 prioritizes repeatability using consistent emission profiles and consistent scan positions across time points to improve trend comparability.

In some embodiments, the analysis unit 30 supports one or more normalization operations 106 to improve comparability across devices and settings. In one embodiment, the normalization operations 106 normalize coherence-related features 35 using a device calibration profile 78. In another embodiment, the normalization operations 106 normalize the tissue-state scalar 37 using a tissue normalization factor 106t derived from baseline distributions for a tissue type and region. In another embodiment, the normalization operations 106 normalize Delta_R values using a baseline dispersion estimate (e.g., standard deviation) to yield a normalized deviation 106n, which may be used as an input to classification policies to improve comparability across cohorts. In some embodiments, the feature extraction module 34 computes coherence-related features 35 from time-segmented data. In one implementation, the preprocessing module 32 divides the sensed signals 23 into multiple segments 107a, processes each segment independently to compute segment features 107f, and then aggregates the segment features to produce the coherence-related features 35. In some embodiments, segmentation reduces sensitivity to transient artifacts, and the confidence module 46 uses segment-to-segment dispersion 107d as an input to the confidence score 47.

In some embodiments, the system supports multiple tissue coupling geometries. In one embodiment, the emission structure 12 and the sensors 22 operate in a reflection geometry 108a in which the sensor subsystem 20 detects a reflected response. In another embodiment, the emission structure 12 and the sensors 22 operate in a transmission geometry 108b in which the sensors detect transmitted and/or scattered components on an opposite side of a tissue region. In another embodiment, the system uses a mixed geometry 108c in which both reflected and transmitted components are measured and combined. In some embodiments, the sensor configuration identifier 22id indicates which geometry is used, and the baseline selection policy 74 selects corresponding baseline entries.

In some embodiments, the system supports multiple patient positioning configurations. In one embodiment, the tissue region 60 is scanned while the patient is seated, and the positioning module 72 uses a handheld guide to control standoff distance. In another embodiment, the tissue region 60 is scanned while the patient is lying on a surface, and the positioning module 72 uses a mechanical mount or gantry. In another embodiment, the system uses wearable alignment features 109a to improve repeatability of scan positions. In some embodiments, a positioning record 109r is stored in the scan metadata 49m to support repeatability in monitoring mode. In some embodiments, the analysis unit 30 supports multi-sensor fusion for a single region-of-interest 62. In one embodiment, the sensor subsystem 20 includes at least two sensors 22 that produce at least two sensor channels 23(1) and 23(2). The feature extraction module 34 computes coherence-related features 35(1) and 35(2) from the sensor channels, and the x-mapping module 36 computes the tissue-state scalar 37 using a fusion mapping 111. In one embodiment, the fusion mapping 111 computes:

x=b1*x1+b2*x2

where x1 and x2 are nonnegative intermediate x values derived from coherence-related features and b1 and b2 are nonnegative fusion weights. In another embodiment, the fusion mapping 111 selects a robust fused value by taking a median across channels. In some embodiments, fusion improves robustness to local sensor noise and improves repeatability.

In some embodiments, the output interface 50 includes a rule-based recommendation generator 113 that outputs a recommended follow-up action 113a. In one embodiment, when the classification output 45 is ABNORMAL and the confidence score 47 exceeds a confidence threshold, the recommendation generator 113 outputs a follow-up recommendation indicating confirmatory testing. In another embodiment, when the classification output 45 is INDETERMINATE, the recommendation generator 113 outputs a recommendation for repeat scanning or for an alternative scan mode. In another embodiment, when the classification output 45 is HEALTHY, the recommendation generator 113 outputs a recommendation for routine monitoring at a periodic interval. In some embodiments, recommended follow-up actions are configurable per protocol and are presented as non-binding guidance for clinician review.

In some embodiments, the system stores an audit log 114 in the output storage 56. In one embodiment, the audit log 114 stores a record of emission profiles used, baseline identifiers selected, classification policies applied, and results generated, enabling traceability of outputs. In some embodiments, the audit log 114 is stored with integrity protection, including a hash 114h and/or digital signature 114s.

In some embodiments, the device includes a software update module 115 configured to update one or more of the emission profiles 13, mapping selection rules 88, baseline databases, and classification policies. In some embodiments, updates are cryptographically signed and verified prior to installation to ensure integrity. In some embodiments, after an update, the calibration workflow 75 is executed and a new calibration profile 78 is stored.

In some embodiments, the recognition metric and deviation computation are implemented so as to preserve explicit mathematical properties that support stable interpretation. In particular, in some embodiments Delta_R is computed as an absolute difference and is therefore nonnegative:

Delta_R>=0

In some embodiments, the recognition metric module 38 computes R_hat as a bounded value in which:

0<=R_hat(x)<1 for x>=0

and R_hat is monotone increasing with x when X_opt>0. In some embodiments, these properties are used as internal sanity checks, where the analysis unit 30 raises an error flag 116e when computed values violate expected bounds, thereby detecting software faults, arithmetic overflows, or invalid input data.

In some embodiments, the system is configured for a variety of clinical applications, including screening, monitoring, and therapy response assessment, by selecting tissue protocols, baselines, and thresholds appropriate to the intended use. The disclosure does not require any particular disease label or any particular clinical claim to be made by the device, and the classification outputs described herein may be configured as risk indicators, screening indicators, monitoring indicators, or other clinical decision support outputs depending on regulatory and clinical context.

The foregoing description provides non-limiting example embodiments with explicit reference numerals to support construction of FIGS. 1-8 and to support later claim drafting. Additional variations may be implemented without departing from the scope of the disclosure, including variations in field type, waveform selection, sensing modality, mapping functions, baseline representation, and decision logic, while maintaining the core computation of a nonnegative tissue-state scalar 37, a bounded recognition metric 39, and a coherence-deviation metric 43 relative to baseline.

In some embodiments, the analysis unit 30 is configured to compute one or more intermediate values used to improve interpretability of the tissue-state scalar 37 and the coherence-deviation metric 43. In one embodiment, the analysis unit 30 computes a coherence feature baseline offset 117 that represents a difference between a measured coherence feature Gamma_measured 117m and a baseline coherence feature Gamma_healthy 117h, and uses the coherence feature baseline offset 117 as an auxiliary output in the output report 49. In some embodiments, the auxiliary output enables a clinician or operator to understand whether a deviation arises primarily from reduced coherence magnitude, increased phase dispersion, altered correlation decay time, or other feature-specific changes.

In some embodiments, the preprocessing module 32 computes a per-acquisition quality score 118q and uses the per-acquisition quality score 118q to gate which segments and/or acquisitions are included in computation of the coherence-related features 35. In one implementation, the preprocessing module 32 assigns a segment quality label 118s to each segment 107a, and the feature extraction module 34 excludes segments having a segment quality label 118s below a minimum acceptable label. In some embodiments, when too few acceptable segments remain, the scan controller 70 automatically triggers the repeat logic step 124.

In some embodiments, the feature extraction module 34 computes coherence-related features 35 using both reference-based and reference-free techniques. In a reference-based technique, the feature extraction module 34 uses the reference channel 28 and computes a reference coherence feature 119r. In a reference-free technique, the feature extraction module 34 computes a self-coherence feature 119s from autocorrelation and/or spectral stability of a single sensor channel. In some embodiments, the x-mapping module 36 selects between reference-based and reference-free features using the feature selection rule 34s, thereby enabling operation in embodiments that omit the reference channel 28.

In some embodiments, the analysis unit 30 computes the tissue-state scalar 37 using a monotone deviation mapping 120m in which larger deviations from baseline produce larger x values. In one embodiment, the analysis unit 30 defines a deviation feature d as:

d=max(0,Gamma_healthy−Gamma_measured)
and computes:

x=d/(Gamma_healthy+epsilon)

where epsilon is a small positive stabilizer constant 120e used to avoid division by zero. In some embodiments, epsilon is stored in the mapping weights 36w and is selected to be small relative to expected baseline coherence feature magnitudes. In some embodiments, the mapping ensures x>=0 and increases with increasing deviation from baseline.

In some embodiments, the analysis unit 30 computes the tissue-state scalar 37 using a log-based deviation mapping 121m for improved dynamic range. In one embodiment:

x=max(0, ln((Gamma_healthy+epsilon)/(Gamma_measured+epsilon)

where epsilon is the stabilizer constant 120e. In some embodiments, the log-based mapping reduces sensitivity to small multiplicative changes and improves comparability across regions that exhibit different baseline coherence magnitudes.

In some embodiments, the recognition metric module 38 computes the recognition metric 39 using X_opt stored as a constant 122x in memory. In one embodiment, the memory 30m stores X_opt as a floating-point constant. In another embodiment, the memory 30m stores X_opt as a rational approximation 122r (e.g., numerator and denominator integers) and the recognition metric module 38 computes R_hat using fixed-point arithmetic. In some embodiments, fixed-point arithmetic reduces numerical drift across device platforms and improves deterministic reproducibility for regulatory and audit purposes.

In some embodiments, the analysis unit 30 includes a bounds-check module 123b that verifies that x satisfies x>=0 and that R_hat_measured satisfies 0<=R_hat_measured<1. In some embodiments, if bounds are violated, the bounds-check module 123b raises the error flag 116e and triggers the scan controller 70 to (i) discard the affected acquisition, (ii) repeat the acquisition, and/or (iii) report a fault condition in the output report 49. In some embodiments, the bounds-check module 123b logs the violated bound and the associated input values in the audit log 114.

In some embodiments, the deviation computation module 42 computes an additional direction indicator 124d indicating whether R_hat_measured is greater than or less than R_hat_healthy. In one embodiment, the deviation computation module 42 computes:

sign_R=sign(R_hat_measured−R_hat_healthy)

and stores sign_R as the direction indicator 124d in the output report 49. In some embodiments, the direction indicator 124d enables distinguishing deviation directionality in embodiments where increases and decreases relative to baseline have distinct clinical interpretation, while still preserving the nonnegative magnitude Delta_R.

In some embodiments, the classification policy 97 includes a tissue-specific threshold table 125t and a tissue-specific margin table 125m. In one embodiment, the threshold table 125t stores thresholds 96 indexed by tissue type identifier 60t and region identifier 60r. In another embodiment, the threshold table 125t stores thresholds indexed additionally by operating mode 18f and/or scan mode 105. In some embodiments, the margin table 125m stores margins 98 that are larger for high-variability regions and smaller for low-variability regions. In some embodiments, the baseline storage 40 stores a baseline variability measure 126v (e.g., standard deviation of baseline R_hat values), and the classification policy 97 derives the margin 98 from the baseline variability measure 126v, thereby normalizing indeterminate bands to expected healthy variability.

In some embodiments, the baseline database is established using a baseline acquisition protocol 127p. In one embodiment, the baseline acquisition protocol 127p specifies an acquisition configuration (emission profile identifier 13id, sensor configuration identifier 22id), a sample count 127n, and cohort metadata 127c for a healthy population. In some embodiments, the baseline acquisition protocol 127p specifies exclusion rules 127e that exclude acquisitions having artifact severity above a threshold or confidence below a threshold. In some embodiments, the baseline storage 40 stores baseline values along with baseline provenance data 127v including acquisition configuration and cohort metadata, enabling later auditing and refinement.

In some embodiments, the baseline storage 40 stores both baseline recognition metrics 41 and baseline feature baselines 128f. In one embodiment, baseline feature baselines 128f include baseline coherence feature values Gamma_healthy 117h for each tissue type and region. In some embodiments, storing baseline feature baselines 128f enables re-computation of x using alternate mappings without requiring re-acquisition of healthy-cohort data, thereby supporting future software updates while preserving baseline consistency.

In some embodiments, the scan controller 70 supports a region alignment routine 129a that improves repeatability of region-of-interest 62 placement. In one embodiment, the region alignment routine 129a uses an alignment marker 129m on a device housing or a patient positioning aid to align scan positions 93 to anatomical landmarks. In another embodiment, the region alignment routine 129a uses a distance sensor 129d and an angle sensor 129g to maintain a target standoff distance and target orientation. In some embodiments, alignment information is stored as alignment metadata 129x in the scan metadata 49m.

In some embodiments, the deviation map 90 is post-processed by a map smoothing module 130s to reduce pixel-level noise. In one embodiment, the map smoothing module 130s applies a spatial filter 130f that averages deviation values 94 across neighboring map regions 92, optionally weighted by region confidence score 102. In some embodiments, the map smoothing module 130s does not alter raw per-region values stored in the output report 49, and instead generates a display-only map 90d for visualization on the display 52.

In some embodiments, the report generation module 48 computes and stores a minimum supporting set of outputs to facilitate later examiner review and enable claim support. In one embodiment, the output report 49 includes: (i) the emission profile identifier 13id; (ii) the sensor configuration identifier 22id; (iii) the mapping identifier 88id indicating which mapping 80, 82, 84, or 86 was used; (iv) the baseline identifier 41id; (v) the computed tissue-state scalar 37; (vi) the computed recognition metric 39; (vii) the computed coherence-deviation metric 43; (viii) the classification output 45; and (ix) the confidence score 47. In some embodiments, storing the mapping identifier 88id and baseline identifier 41id supports reproducibility and traceability. In some embodiments, the device includes an operator override control 1310 that allows an operator to select a scan mode 105, select a tissue protocol 91, and/or select whether to use population baseline or patient-specific baseline when both are available. In some embodiments, the operator override control 1310 is logged in the audit log 114 along with the reason code 131r (e.g., screening, follow-up, monitoring) to support later review.

In some embodiments, the system includes a secure data module 132s that encrypts stored output reports 49 and audit logs 114 in the output storage 56. In one embodiment, the secure data module 132s uses an encryption key 132k stored in secure hardware and enforces access control via an authentication module 132a. In some embodiments, access control prevents unauthorized modification of baselines, thresholds, mapping rules, and stored diagnostic reports.

In some embodiments, the system is configured for different deployment environments without changing the core computational outputs. In one embodiment, a point-of-care deployment uses a portable housing with the output interface 50 providing immediate results. In another embodiment, a clinical deployment uses a workstation interface receiving results via the communication interface 54. In another embodiment, a remote deployment transmits results to the external computing system 57 for clinician review. In each embodiment, the core steps include emission of recognition field 11, sensing of tissue response signals 21 to produce sensed signals 23, computation of tissue-state scalar 37, computation of recognition metric 39, computation of coherence-deviation metric 43 relative to baseline, and output of classification output 45.

In some embodiments, a method of non-invasive medical diagnostics is implemented using the elements described herein. In one example method, the method comprises: generating, by the field emitter subsystem 10, the recognition field 11 according to the emission profile 13; sensing, by the sensor subsystem 20, the tissue response signals 21 associated with the recognition field 11 to produce the sensed signals 23; preprocessing, by the preprocessing module 32, the sensed signals 23 to produce conditioned signals 33; extracting, by the feature extraction module 34, coherence-related features 35 from the conditioned signals 33; computing, by the x-mapping module 36, the tissue-state scalar 37 as a nonnegative value; computing, by the recognition metric module 38, the recognition metric 39 using R_hat(x)=x/(x+X_opt); retrieving, from the baseline storage 40, the baseline recognition metric 41; computing, by the deviation computation module 42, the coherence-deviation metric 43 as Delta_R=abs(R_hat_measured−R_hat_healthy); and outputting, by the output interface 50, at least one of the coherence-deviation metric 43 and the classification output 45.

In some embodiments, the method further comprises generating the deviation map 90 by repeating the method across multiple scan positions 93 and associating deviation values 94 with corresponding map regions 92. In some embodiments, the method further comprises triggering the repeat logic step 124 when an INDETERMINATE classification output is produced or when a confidence score falls below a threshold, and repeating at least a portion of the method for a same region-of-interest 62 and/or for an alternative emission profile.

The foregoing embodiments and method descriptions are intended to provide an enabling disclosure for examiner review and prosecution and are not limiting. Variations may be implemented in field type, waveform, sensing modality, preprocessing, coherence feature computation, nonnegative mapping to x, baseline representation, and decision logic, while maintaining the core computation of the bounded recognition metric and baseline-referenced coherence deviation.

In some embodiments, the field emitter subsystem 10 and the sensor subsystem 20 are configured to operate in a closed-loop measurement mode 133 in which at least one parameter of the emission profile 13 is adapted based on measured tissue response. In one embodiment, the analysis unit 30 computes a response stability metric 133s from the conditioned signals 33, and the scan controller 70 adjusts at least one emission parameter (e.g., amplitude 13g, modulation frequency 13b, duty cycle 13f, dwell time 73d, or a band selection parameter 13c) to improve stability, improve SNR, or reduce artifact sensitivity. In some embodiments, the closed-loop measurement mode 133 uses a target stability threshold 133t, and the scan controller 70 continues emission adjustments until the response stability metric 133s satisfies the target stability threshold 133t or until a maximum adjustment budget 133b is reached.

In some embodiments, the system supports a differential measurement mode 134 in which at least two acquisitions are performed under different conditions and the analysis unit 30 computes a differential deviation 134d. In one embodiment, the scan controller 70 performs a first acquisition using a first emission profile 13(1) and a second acquisition using a second emission profile 13(2), and the analysis unit 30 computes Delta_R(1) and Delta_R(2) and computes: Delta_R_diff=abs(Delta_R(1)−Delta_R(2))

In some embodiments, the classification module 44 uses Delta_R_diff as an additional input to classification, such as requiring that Delta_R(1) and Delta_R(2) both exceed a threshold or requiring that Delta_R_diff exceed a differential threshold for certain tissue protocols.

In some embodiments, the system supports a paired-region comparison mode 135 in which a first tissue region 60(1) and a second tissue region 60(2) are scanned and compared. In one embodiment, the paired regions correspond to bilateral anatomy (e.g., left/right), proximal/distal regions, or a target region and a nearby reference region. In some embodiments, the analysis unit 30 computes R_hat_measured (1) and R_hat_measured (2) and computes a paired deviation metric 135p as:

Delta_R_pair=abs(R_hat_measured(1)−R_hat_measured(2))

In some embodiments, the paired deviation metric 135p is used in addition to baseline-referenced Delta_R to reduce sensitivity to patient-wide factors and to highlight asymmetric deviations.

In some embodiments, the system supports a tissue coupling compensation mode 136 in which the analysis unit 30 compensates for variability in coupling between the emission structure 12 and the tissue region 60. In one embodiment, the coupling interface 12a includes a pressure sensor 136p and/or a proximity sensor 136d that produces coupling metadata 136m, and the preprocessing module 32 uses coupling metadata 136m to normalize amplitude-dependent features while retaining coherence-related features 35. In some embodiments, coupling compensation improves repeatability across operators and across repeated sessions.

In some embodiments, the feature extraction module 34 computes a multi-band feature set 137 in which coherence-related features 35 are computed over multiple bands B1, B2, and B3. In one embodiment, the multi-band feature set 137 includes Gamma (B1) 137a, Gamma (B2) 137b, and Gamma (B3) 137c. In some embodiments, the x-mapping module 36 computes nonnegative intermediate values x1, x2, and x3 for the bands and computes:

x=a1*x1+a2*x2+a3*x3

where a1, a2, and a3 are nonnegative weights stored as mapping weights 36w. In some embodiments, multi-band computation provides robustness to tissue depth variation and to heterogeneity of tissue composition.

In some embodiments, the system supports a region segmentation rule 138 that defines how scan positions 93 are grouped into map regions 92. In one embodiment, the region segmentation rule 138 defines fixed map tiles of equal size. In another embodiment, the region segmentation rule 138 defines anatomically shaped regions based on a template. In another embodiment, the region segmentation rule 138 defines adaptive regions that expand or contract based on confidence and deviation gradients. In some embodiments, the report generation module 48 stores the region segmentation rule identifier 138id as part of scan metadata 49m to support reproducibility. In some embodiments, the device includes an initialization routine 139 executed at power-on or prior to scanning. In one embodiment, the initialization routine 139 includes a sensor check 139s verifying sensor noise floor and saturation headroom, an emitter check 139e verifying emitted power estimate 18d and waveform integrity, and a timing check 139t verifying synchronization between reference channel 28 and sensed signals 23. In some embodiments, the initialization routine 139 sets a ready flag 139r and the output interface 50 presents a ready indicator 139i only when the ready flag 139r is set.

In some embodiments, the analysis unit 30 includes a configuration memory 140m storing device configuration parameters 140p. In one embodiment, the device configuration parameters 140p include a device identifier 140id, a calibration profile identifier 78id, an emission profile library identifier 131ib, and permitted scan modes 105. In some embodiments, the configuration memory 140m also stores software version information 140v, and the report generation module 48 includes the software version information 140v in scan metadata 49m for traceability during prosecution and later device deployment.

In some embodiments, the system supports a clinician review workflow 141 in which results are reviewed and optionally annotated. In one embodiment, the external computing system 57 provides the clinician review interface 57c that allows a clinician to view the deviation map 90, view region confidence scores 102, and view scan metadata 49m. In some embodiments, the clinician review interface 57c allows entry of a clinician annotation 141a and a confirmation label 141c (e.g., confirmed healthy, confirmed abnormal, uncertain). In some embodiments, the confirmation label 141c may be used by the baseline update policy 104 and/or by threshold refinement workflows, subject to protocol controls.

In some embodiments, the system supports a threshold refinement workflow 142 in which thresholds 96 are updated based on validated outcomes. In one embodiment, the threshold refinement workflow 142 aggregates de-identified deviation values 94 with outcome labels and computes updated thresholds 96u and updated margins 98u for one or more tissue protocols. In some embodiments, updated thresholds 96u are installed via the software update module 115 and stored as updated classification policy 97u in the baseline storage 40. In some embodiments, thresholds are updated only when update integrity checks pass, including signature verification and calibration checks.

In some embodiments, the device includes a local storage limit policy 143 that controls how many output reports 49 and audit logs 114 are retained in the output storage 56. In one embodiment, when storage reaches a limit, the system prompts export via the communication interface 54. In another embodiment, the system uses a secure rolling buffer, deleting oldest records after successful export and integrity confirmation. In some embodiments, the audit log 114 retains baseline identifiers 41id, mapping identifiers 88id, and classification policy identifiers 97id to support future examiner questions about how outputs are derived.

In some embodiments, the disclosure includes a non-transitory computer-readable medium 30c storing instructions that, when executed by the processor 30p, cause the processor 30p to perform operations comprising: controlling the field emitter subsystem 10 to generate the recognition field 11 according to the emission profile 13; receiving the sensed signals 23 from the sensor subsystem 20; computing coherence-related features 35; computing the tissue-state scalar 37 as a nonnegative value; computing the recognition metric 39 using R_hat(x)=x/(x+X_opt); retrieving a baseline recognition metric 41 from baseline storage 40; computing the coherence-deviation metric 43 as Delta_R=abs(R_hat_measured−R_hat_healthy); and outputting at least one of the coherence-deviation metric 43 and the classification output 45 using the output interface 50.

In some embodiments, the disclosure includes an apparatus embodiment in which the analysis unit 30, the baseline storage 40, and the output interface 50 are integrated into a handheld housing 144h. In such embodiments, the handheld housing 144h may include a power source 144p, a control button 144b, and the display 52. In some embodiments, the emission structure 12 is integrated at a distal end of the handheld housing 144h and the sensors 22 are arranged adjacent the emission structure 12 to support a compact reflection geometry. In other embodiments, the disclosure includes a stationary embodiment in which the positioning module 72 includes a mechanical arm 145a or a gantry 145g configured to position the emission structure 12 and/or sensors 22 at defined scan positions 93 for mapping a larger tissue region.

In some embodiments, the disclosed framework is implemented without requiring that any particular physiological interpretation be assigned to the computed tissue-state scalar 37. In such embodiments, x is an engineered nonnegative scalar derived from coherence-related features that is used to compute a bounded recognition metric 39 and a baseline-referenced coherence-deviation metric 43. This allows the device to function as a screening and monitoring tool across multiple tissue protocols while maintaining stable bounded metrics that support calibration and longitudinal trending.

In some embodiments, the disclosure includes examples of key mathematical invariants used for internal checks and robust computation. In particular, Delta_R is nonnegative by construction as an absolute value, and R_hat(x) is bounded and monotone for x>=0 and X_opt>0. In some embodiments, the analysis unit 30 stores an invariant check record 146i in the audit log 114 indicating whether bounds and monotonicity checks passed for a given scan session. In some embodiments, invariant checks support robustness and reproducibility of computed outputs for examiner review, prosecution, and later device implementation.

The foregoing description of embodiments is intended to be illustrative and not limiting. A person of ordinary skill in the art will recognize that variations may be made in emitter implementation, waveform selection, sensor modality, coherence feature computation, mapping to a nonnegative tissue-state scalar, baseline selection and representation, classification policy, and scan workflow, while maintaining the core operations of generating a recognition field 11, sensing tissue response signals 21, computing a bounded recognition metric 39, computing a coherence-deviation metric 43 relative to a baseline representation, and outputting one or more diagnostic outputs.

What is claimed:

1. A non-invasive diagnostic system comprising:
a field emitter configured to generate a recognition field according to an emission profile;
a sensor subsystem configured to detect a tissue response signal associated with interaction between the recognition field and biological tissue; and
at least one processor coupled to the sensor subsystem and configured to:
derive, from the tissue response signal, one or more coherence-related features;
compute a nonnegative tissue-state scalar x from the one or more coherence-related features;
compute a recognition metric R_hat_measured using a recognition operator R_hat(x)=x/(x+X_opt), where X_opt is a predetermined constant;
retrieve or determine a baseline recognition metric R_hat_healthy associated with healthy tissue;
compute a coherence-deviation metric Delta_R as an absolute value of (R_hat_measured−R_hat_healthy); and
output at least one of (i) the coherence-deviation metric or (ii) a classification output based on the coherence-deviation metric.

2. The system of claim 1, wherein the predetermined constant X_opt is defined by:
phi=(1+sqrt(5))/2, and X_opt=phi/pi.

3. The system of claim 1, wherein computing the nonnegative tissue-state scalar x comprises computing x using x=(1/Gamma)−1, where Gamma is a coherence feature in (0, 1].

4. The system of claim 1, wherein computing the nonnegative tissue-state scalar x comprises computing x using x=−ln(Gamma), where Gamma is a coherence feature in (0, 1].

5. The system of claim 1, wherein computing the nonnegative tissue-state scalar x comprises computing x using x=max(0, (Gamma_healthy−Gamma_measured)/Gamma_healthy), where Gamma_measured is computed from the tissue response signal and Gamma_healthy is associated with healthy tissue.

6. The system of claim 1, wherein deriving the one or more coherence-related features comprises computing a magnitude-squared coherence between a reference signal associated with the emission profile and a measured signal associated with the tissue response signal.

7. The system of claim 1, wherein the at least one processor stores or retrieves a threshold and a margin, and is configured to compute the classification output using:
ABNORMAL when Delta_R is greater than threshold plus margin;
HEALTHY when Delta_R is less than threshold minus margin; and
INDETERMINATE when Delta_R is between threshold minus margin and threshold plus margin.

8. The system of claim 1, wherein the baseline recognition metric is selected from a baseline database indexed by at least one of tissue type, anatomical region, cohort metadata, emission profile identifier, or sensor configuration identifier.

9. The system of claim 1, wherein the baseline recognition metric is a patient-specific baseline derived from a prior scan of a same patient.

10. The system of claim 1, wherein the at least one processor is configured to compute the coherence-deviation metric for a plurality of regions-of-interest and generate a deviation map associating respective regions-of-interest with respective coherence-deviation metric values.

11. The system of claim 1, wherein the at least one processor is configured to compute a confidence score based on at least one of signal-to-noise ratio, artifact detection output, environmental noise estimate, or repeatability across repeated acquisitions, and to output the confidence score with the classification output.

12. The system of claim 11, wherein the at least one processor is configured to trigger a repeat acquisition when the classification output is INDETERMINATE or when the confidence score is below a confidence threshold.

13. The system of claim 1, wherein the emission profile comprises at least one of a carrier frequency, a modulation frequency, a bandwidth, a pulse width, a repetition rate, a duty cycle, an amplitude, or a phase code.

14. The system of claim 1, wherein the field emitter is configured to operate at an effective operating frequency in a permitted device band that is deterministically mapped from a recognition-referenced parameter derived from X_opt, using at least one of scaling, mixing, frequency division, frequency multiplication, harmonic selection, or band-limited approximation to define at least one carrier and/or modulation component of the emission profile.

15. A non-invasive diagnostic method comprising:
generating a recognition field according to an emission profile; sensing a tissue response signal associated with interaction between the recognition field and biological tissue; deriving one or more coherence-related features from the tissue response signal; computing a nonnegative tissue-state scalar x from the one or more coherence-related features; computing a recognition metric R_hat_measured using a recognition operator R_hat(x)=x/(x+X_opt), where X_opt is a predetermined constant; retrieving or determining a baseline recognition metric R_hat_healthy associated with healthy tissue; computing a coherence-deviation metric Delta_R as an absolute value of (R_hat_measured−R_hat_healthy); and outputting at least one of (i) the coherence-deviation metric or (ii) a classification output based on the coherence-deviation metric.

16. The method of claim 15, further comprising storing or retrieving a threshold and a margin, and computing the classification output using:
ABNORMAL when Delta_R is greater than threshold plus margin;
HEALTHY when Delta_R is less than threshold minus margin; and
INDETERMINATE when Delta_R is between threshold minus margin and threshold plus margin.

17. The method of claim 15, further comprising computing the coherence-deviation metric for a plurality of regions-of-interest and generating a deviation map associating respective regions-of-interest with respective coherence-deviation metric values.

18. The method of claim 15, further comprising:
computing a confidence score based on at least one of signal-to-noise ratio, artifact detection output, environmental noise estimate, or repeatability across repeated acquisitions;
outputting the confidence score with the classification output; and
repeating at least one of the generating or sensing steps when (i) the classification output is INDETERMINATE or (ii) the confidence score is below a confidence threshold.

19. A non-transitory computer-readable medium storing instructions that, when executed by at least one processor of a diagnostic system, cause the diagnostic system to perform operations comprising:
receiving a tissue response signal detected in association with a recognition field generated according to an emission profile;
deriving one or more coherence-related features from the tissue response signal;
computing a nonnegative tissue-state scalar x from the one or more coherence-related features;
computing a recognition metric R_hat_measured using a recognition operator R_hat(x)=x/(x+X_opt), where X_opt is a predetermined constant;
retrieving or determining a baseline recognition metric R_hat_healthy associated with healthy tissue;
computing a coherence-deviation metric Delta_R as an absolute value of (R_hat_measured-R_hat_healthy); and
outputting at least one of (i) the coherence-deviation metric or (ii) a classification output based on the coherence-deviation metric.

20. The non-transitory computer-readable medium of claim 19, wherein the operations further comprise generating a deviation map by computing the coherence-deviation metric for a plurality of regions-of-interest and associating respective regions-of-interest with respective coherence-deviation metric values.
